(19) United States
(12) Patent Application Publication
(10) Pub. No.: US 2024/0412812 A1
(43) Pub. Date: Dec. 12, 2024

(54) METHODS AND SYSTEMS FOR DETECTING AND REMOVING CONTAMINATION FOR COPY NUMBER ALTERATION CALLING

(71) Applicant: Foundation Medicine, Inc., Boston, MA (US)

(72) Inventors: Jason D. HUGHES, Providence, RI (US); Justin NEWBERG, Cambridge, MA (US)

(73) Assignee: Foundation Medicine, Inc., Boston, MA (US)

(21) Appl. No.: 18/698,747

(22) PCT Filed: Oct. 7, 2022
(86) PCT No.: PCT/US2022/077800

§ 371 Date: Apr. 4, 2024

Related U.S. Application Data
(60) Provisional application No. 63/253,912, filed on Oct. 8, 2021.

(51) Int. Cl. G16B 20/10; G16B 20/20

(52) U.S. Cl. CPC G16B 20/10; G16B 20/20

(57) ABSTRACT

Methods and systems for performing iterative contamination detection and segmentation of sequence read data are described. The methods are based on comparing a distribution of minor allele frequencies (MAPs) for a plurality of single nucleotide polymorphisms (SNPs) detected in the sample to an expected distribution of minor allele frequencies for a plurality of selected SNP loci, and adjusting a MAP threshold used to discriminate between aberrant SNPs (SNPs exhibiting a different distribution of MAP values than that expected for the plurality of selected SNPs) and those conforming to the expected distribution of minor allele frequencies for the plurality of selected SNP loci. The methods may be used to estimate the degree of contamination in a sample and to provide segmentation of sequence read data for the sample, and may further comprise building a copy number model that predicts a copy number for one or more gene loci.

### CROSS-REFERENCE TO RELATED APPLICATIONS

This application claims the priority benefit of U.S. Provisional Patent Application Ser. No. 63/253,912, filed Oct. 8, 2021, the contents of which are incorporated herein by reference in their entirety.

### FIELD OF THE INVENTION

The present disclosure relates generally to methods and systems for analyzing genomic profiling data, and more specifically to methods and systems for contamination detection and segmentation of sequence read data used for automated calling of copy number alterations.

### BACKGROUND

Structural variants (SVs) are large genomic alterations that typically comprise alterations of at least 50 base pairs (bp) in length (Mahmoud, et al. (2019), “Structural variant calling: the long and the short of it”, Genome Biology 20:246). These large genomic alterations can be classified as deletions, duplications, insertions, inversions, and translocations, and describe different combinations of DNA gains, losses, or rearrangements.

Copy number alterations (CNAs) (also referred to as copy number variations (CNVs)) are a subtype of large structural variants that primarily comprise deletions or duplications, and may encompass alterations of up to half a million nucleotides in length. Somatic copy number variations (CNVs) can play a crucial role in the development of many types of cancer (Samadian, et al. (2018), “Bamgineer: Introduction of simulated allele-specific copy number variants into exome and targeted sequence data sets”, PLOS Comput Biol. 14(3):e1006080). The development of next-generation sequencing (NGS) methods have enabled the development of algorithms to computationally infer CNA profiles from a variety of sequencing data sets, including exome and targeted sequence data.

However, existing methods for detecting and calling CNAs based on sequencing data are prone to error due to sample contamination and segmentation errors. Human contamination (i.e., contamination by DNA not arising from the subject) is a common issue with tumor samples (found in about 1-5% of samples to be analyzed), usually at relatively low contamination levels (<5% contamination by non-subject DNA). The presence of contamination in the sample can lead to erroneous detection and calling of variant sequences in the sample, and contribute to modeling error when attempting to detect and call copy number alterations. For example, contaminated patient samples can appear to be very high purity (high tumor fraction) samples due to the presence of low-frequency SNPs that didn't actually arise from the patient sample. Thus there is a need for improved methods for detecting contamination in sequence read data and for removing the contaminating sequence data from segmentation and copy number modeling.

### BRIEF SUMMARY OF THE INVENTION

Methods and systems for performing iterative contamination detection and segmentation of sequence read data. The methods comprise estimating a degree of contamination for the sample based on a distribution of allele frequencies (e.g., minor allele frequencies) for a selected set of single nucleotide polymorphisms (SNPs) (e.g., heterozygous single nucleotide polymorphisms (SNPs)). Then, using the estimated degree of contamination as an initial value for a first threshold (e.g., a minor allele frequency (MAF) threshold), the sequencing data is iteratively segmented while simultaneously excluding sequencing data from the segmentation process that comprises SNPs having allele frequencies that are below the first threshold. At each iteration, the remaining SNPs are classified as aberrant (i.e., likely due to contamination) if they have an allele frequency that is different from the allele frequencies for other SNPs detected on the same segment, and the first threshold is incrementally adjusted based a comparison of the distribution of aberrant SNP allele frequencies to the expected distribution of allele frequencies for the selected set of, e.g., heterozygous SNPs. The segmenting, classifying, and first threshold adjusting steps are repeated each time the first threshold is increased. When no further increase of the first threshold is required (or there is no further change in the distribution of aberrant SNP allele frequencies, or a specified maximum number of iterations has been reach), the segmentation data and an estimated degree of contamination for the sample (equal to the final value of the first threshold) is output. In some instances, the method further comprises using the segmentation data and estimated degree of contamination to build a copy number model that predicts a copy number for the one or more gene loci.

Disclosed herein are methods comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules, wherein one or more of the plurality of sequencing reads overlap one or more gene loci within one or more subgenomic intervals in the sample; receiving, at one or more processors, sequence read data for the plurality of sequence reads; estimating, using the one or more processors, a degree of contamination for the sample based on a distribution of allele frequencies (AFs) for a plurality of selected single nucleotide polymorphisms (SNPs) identified within a plurality of gene loci in the sequence read data; segmenting, using the one or more processors, the sequence read data into two or more segments, wherein each segment has a same copy number, and wherein sequence read data comprising SNPs that exhibit an allele frequency below a first threshold are excluded from the segmenting process; classifying, using the one or more processors, a SNP detected on a segment of the two or more segments as aberrant when the SNP exhibits an allele frequency that is different from an allele frequency for other SNPs detected on the same segment; adjusting, using the one or more processors, the first threshold based on a distribution of aberrant SNP allele frequencies; repeating the segmenting, classifying, and adjusting steps when the first threshold is increased; and outputting, using the one or more processors, the segmentation data and a final threshold as the estimated degree of contamination for the sample.

In some embodiments, the method further comprises setting an initial value for the first threshold as equal to the estimated degree of contamination for the sample. In some embodiments, the plurality of selected single nucleotide polymorphisms (SNPs) comprises a plurality of selected heterozygous single nucleotide polymorphisms (SNPs). In some embodiments, the predetermined distribution of allele frequencies (AFs) for the plurality of selected single nucleotide polymorphisms (SNPs) comprises a predetermined distribution of minor allele frequencies (MAFs) for the plurality of selected single nucleotide polymorphisms (SNPs). In some embodiments, the method further comprises using the segmentation data and estimated degree of contamination output by the one or more processors to build a copy number model that predicts a copy number for the one or more gene loci. In some embodiments, the method further comprises excluding all sequence read data for gene loci on a same segment as SNPs that exhibit an allele frequency below the final threshold from a copy number analysis for the one or more gene loci. In some embodiments, estimating the degree of contamination for the sample based on the distribution of allele frequencies for the plurality of selected SNPs comprises determining a percentage of the heterozygous SNPs identified in the sample that have MAFs that differ from an expected allele frequency distribution for a plurality of selected heterozygous SNPS identified within the plurality of gene loci by at least a second threshold. In some embodiments, a SNP is classified as aberrant when the SNP exhibits an allele frequency that is different from the allele frequency for other SNPs detected on the same segment based on an absolute value of the difference in allele frequency. In some embodiments, a SNP is classified as aberrant if it exhibits an allele frequency that is different from the allele frequency for other SNPS detected on the same segment based on a statistical analysis. In some embodiments, the segmenting is performed using a circular binary segmentation (CBS) method, a maximum likelihood method, a hidden Markov chain method, a walking Markov method, a Bayesian method, a long-range correlation method, or a change point method. In some embodiments, the segmenting is performed using a change point method, and the change point method is a pruned exact linear time (PELT) method. In some embodiments, the first threshold is incrementally adjusted to reduce a number of SNPs classified as aberrant, and wherein the first threshold is set based on a percentage of SNPs identified in the sample that have allele frequencies that differ from an expected allele frequency distribution for a plurality of selected heterozygous SNPS identified within the plurality of gene loci by at least a third threshold. In some embodiments, the subject is suspected of having or is determined to have a disease. In some embodiments, the disease is cancer. In some embodiments, the method is used as part of a copy number alteration (CNA) calling pipeline for routine testing. In some embodiments, the method is used as part of a copy number alteration (CNA) calling pipeline for prenatal testing. In some embodiments, the method further comprises collecting the sample from the subject. In some embodiments, the sample comprises a tissue biopsy sample, a liquid biopsy sample, or a normal control. In some embodiments, the sample is a tissue biopsy sample and comprises bone marrow. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the next generation sequencing (NGS) comprises paired end sequencing. In some embodiments, the sequencer comprises a next generation sequencer. In some embodiments, the method further comprises generating, by the one or more processors, a report indicating the predicted copy number for the one or more gene loci. In some embodiments, the method further comprises transmitting the report to a healthcare provider. In some embodiments, the report is transmitted via a computer network or a peer-to-peer connection.

Disclosed herein are methods for detecting contamination in sequence read date for a sample from a subject, the method comprising: receiving, at one or more processors, sequence read data for a plurality of sequence reads; estimating, using the one or more processors, a degree of contamination for the sample based on a distribution of allele frequencies (AFs) for a plurality of selected single nucleotide polymorphisms (SNPs) identified within a plurality of gene loci in the sequence read data; segmenting, using the one or more processors, the sequence read data into two or more segments, wherein each segment has a same copy number, and wherein sequence read data comprising SNPs that exhibit an allele frequency below a first threshold are excluded from the segmenting process; classifying, using the one or more processors, a SNP detected on a segment of the two or more segments as aberrant if the SNP exhibits an allele frequency that is different from an allele frequency for other SNPs detected on the same segment; adjusting, using the one or more processors, the first threshold based on a distribution of aberrant SNP allele frequencies; repeating the segmenting, classifying, and adjusting steps when the first threshold is increased; and outputting, using the one or more processors, the segmentation data and a final threshold as the estimated degree of contamination for the sample.

In some embodiments, one or more of the plurality of sequence reads overlap one or more gene loci within one or more subgenomic intervals in the sample.

In some embodiments, the method further comprises setting an initial value for the first threshold as equal to the estimated degree of contamination for the sample.

In some embodiments, the plurality of selected single nucleotide polymorphisms (SNPs) comprises a plurality of selected heterozygous single nucleotide polymorphisms (SNPs).

In some embodiments, the predetermined distribution of allele frequencies (AFs) for the plurality of selected single nucleotide polymorphisms (SNPs) comprises a predetermined distribution of minor allele frequencies (MAFs) for the plurality of selected single nucleotide polymorphisms (SNPs).

In some embodiments, the method further comprises using the segmentation data and estimated degree of contamination output by the one or more processors to build a copy number model that predicts a copy number for the one or more gene loci. In some embodiments, the method further comprises excluding all sequence read data for SNPs that exhibit an allele frequency below the final threshold from a copy number analysis for the one or more gene loci. In some embodiments, the method further comprises excluding all sequence read data for gene loci on a same segment as SNPs that exhibit an allele frequency below the final threshold from a copy number analysis for the one or more gene loci.

In some embodiments, the plurality of selected SNPs identified within the plurality of gene loci comprises at least 100 SNP loci. In some embodiments, the plurality of selected SNPs identified within the plurality of gene loci comprises at least 1,000 SNPs. In some embodiments, the plurality of selected SNPs identified within the plurality of gene loci comprises at most 10,000 SNP loci. In some embodiments, the plurality of selected SNPs identified within the plurality of gene loci comprises at most 100,000 SNP loci. In some embodiments, the plurality of selected SNPs identified within the plurality of gene loci comprise at most 1,000,000 SNP loci.

In some embodiments, the plurality of selected single nucleotide polymorphisms (SNPs) identified within a plurality of gene loci comprises biallelic heterozygous SNPs having unbiased heterozygous allele frequencies of about 50%. In some embodiments, the plurality of selected single nucleotide polymorphisms (SNPs) identified within a plurality of gene loci comprises biallelic heterozygous SNPs having reference and alternate alleles that are observed at greater than 20% global allele frequency. In some embodiments, the plurality of selected single nucleotide polymorphisms (SNPs) identified within a plurality of gene loci comprises biallelic heterozygous SNPs having reference and alternate alleles that are observed at greater than 20% global MAF.

In some embodiments, estimating the degree of contamination for the sample based on the distribution of allele frequencies for the plurality of selected SNPs comprises determining a percentage of heterozygous SNPs identified in the sample that have allele frequencies that differ from an expected allele frequency distribution for a plurality of selected heterozygous SNPs identified within the plurality of gene loci by at least a second threshold.

In some embodiments, the sequence read data is converted to log 2 coverage ratio data prior to performing the segmenting step.

In some embodiments, a SNP is classified as aberrant when the SNP exhibits an allele frequency that is different from the allele frequency for other SNPs detected on the same segment based on an absolute value of the difference in allele frequency. In some embodiments, a SNP is classified as aberrant when the SNP exhibits an allele frequency that is different from the allele frequency for other SNPS detected on the same segment based on a statistical analysis. In some embodiments, the statistical analysis comprises a t-test.

In some embodiments, the segmenting is performed using a circular binary segmentation (CBS) method, a maximum likelihood method, a hidden Markov chain method, a walking Markov method, a Bayesian method, a long-range correlation method, or a change point method. In some embodiments, the segmenting is performed using a change point method, and the change point method is a pruned exact linear time (PELT) method.

In some embodiments, the segmenting, classifying, and adjusting steps are repeated for up to 1 to 10 iterations.

In some embodiments, the first threshold is incrementally adjusted to reduce a number of SNPs classified as aberrant, and wherein the first threshold is set based on a percentage of SNPs identified in the sample that have allele frequencies that differ from an expected allele frequency distribution for a plurality of selected heterozygous SNPs identified within the plurality of gene loci by at least a third threshold.

In some embodiments, a limit of detection for detecting contamination in the sample is less than about 10%. In some embodiments, a limit of detection for detecting contamination in the sample is less than about 5%. In some embodiments, a limit of detection for detecting contamination in the sample is less than about 1%. In some embodiments, a limit of detection for detecting contamination in the sample is less than about 0.5%.

In some embodiments, the first threshold has a value of 0.2, 0.3, 0.4, or 0.5.

In some embodiment, the second threshold is at least 1, at least 2, at least 3, or at least 4 standard deviations from the mean of the expected allele frequency distribution for the plurality of selected heterozygous SNPs.

In some embodiments, the third threshold is at least 1, at least 2, at least 3, or at least 4 standard deviations from the mean of the expected allele frequency distribution for the plurality of selected heterozygous SNPs.

Also disclosed herein are methods for calling copy number alterations (CNAs) in a sample from a subject, comprising: receiving, at one or more processors, sequence read data for a plurality of sequence reads; estimating, using the one or more processors, a degree of contamination for the sample based on a distribution of allele frequencies (AFs) for a plurality of selected single nucleotide polymorphisms (SNPs) identified within a plurality of gene loci in the sequence read data; segmenting, using the one or more processors, the sequence read data into two or more segments, wherein each segment has a same copy number, and wherein sequence read data comprising SNPs that exhibit an allele frequency below a first threshold are excluded from the segmenting process; classifying, using the one or more processors, a SNP detected on a segment of the two or more segments as aberrant when the SNP exhibits an allele frequency that is different from an allele frequency for other SNPs detected on the same segment; adjusting, using the one or more processors, the first threshold based on a distribution of aberrant SNP allele frequencies; repeating the segmenting, classifying, and adjusting steps when the first threshold is increased; outputting, using the one or more processors, the segmentation data and a final threshold as the estimated degree of contamination for the sample; using the segmentation data and the estimated degree of contamination output by the one or more processors to build a copy number model that predicts a copy number for the one or more gene loci; and calling a copy number alterations for the one or more gene loci.

In some embodiments, one or more of the plurality of sequence reads overlap one or more gene loci within one or more subgenomic intervals in the sample.

In some embodiments, the method further comprises setting an initial value for the first threshold as equal to the estimated degree of contamination for the sample.

In some embodiments, the plurality of selected single nucleotide polymorphisms (SNPs) comprises a plurality of selected heterozygous single nucleotide polymorphisms (SNPs).

In some embodiments, the predetermined distribution of allele frequencies (AFs) for the plurality of selected single nucleotide polymorphisms (SNPs) comprises a predetermined distribution of minor allele frequencies (MAFs) for the plurality of selected single nucleotide polymorphisms (SNPs).

In some embodiments, the called CNAs for the one or more gene loci are used to diagnose or confirm a diagnosis of disease in the subject. In some embodiments, the disease is cancer. In some embodiments, the method further comprises selecting an anti-cancer therapy to administer to the subject based on the called CNAs for the one or more gene loci. In some embodiments, the method further comprises determining an effective amount of the anti-cancer therapy to administer to the subject based on the called CNAs for the one or more gene loci. In some embodiments, the method further comprising administering the anti-cancer therapy to the subject based on the called CNAs for the one or more gene loci. In some embodiments, the anti-cancer therapy comprises chemotherapy, radiation therapy, immunotherapy, a targeted therapy, or surgery. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

I some embodiments, the one or more gene loci comprise between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

Disclosed herein are methods for diagnosing a disease, the method comprising: diagnosing that a subject has the disease based on called CNAs for a sample from the subject, wherein the called CNAs are determined according to any of the methods disclosed herein.

Disclosed herein are methods of selecting an anti-cancer therapy, the method comprising: responsive to calling CNAs for one or more gene loci for a sample from a subject, selecting an anti-cancer therapy for the subject, wherein the called CNAs are is determined according to any of the methods disclosed herein.

Disclosed herein are methods of treating a cancer in a subject, comprising: responsive to calling CNAs for one or more gene loci for a sample from the subject, administering an effective amount of an anti-cancer therapy to the subject, wherein the called CNAs are determined according to any of the methods disclosed herein.

Disclosed herein are methods for monitoring tumor progression or recurrence in a subject, the method comprising: calling CNAs for one or more gene loci in a first sample obtained from the subject at a first time point according to any of the methods disclosed herein; calling CNAs for one or more gene loci in a second sample obtained from the subject at a second time point; and comparing the first called CNAs to the second called CNAs for the one or more gene loci, thereby monitoring the tumor progression or recurrence. In some embodiments, the called CNAs for the one or more gene loci in the second sample is determined according to any of the methods disclosed herein. In some embodiments, the method further comprises adjusting an anti-cancer therapy in response to the tumor progression. In some embodiments, the method further comprises adjusting a dosage of the anti-cancer therapy or selecting a different anti-cancer therapy in response to the tumor progression. In some embodiments, the method further comprises administering the adjusted anti-cancer therapy to the subject. In some embodiments, the first time point is before the subject has been administered an anti-cancer therapy, and wherein the second time point is after the subject has been administered the anti-cancer therapy. In some embodiments, the subject has a cancer, is at risk of having a cancer, is being routine tested for cancer, or is suspected of having a cancer. In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments, any of the methods disclosed herein may further comprise determining, identifying, or applying the called CNAs for the one or more gene loci in the sample as a diagnostic value associated with the sample. In some embodiments, any of the methods disclosed herein may further comprise generating a genomic profile for the subject based on the called CNAs for the one or more gene loci. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test. In some embodiments, the method further comprises selecting an anti-cancer agent, administering an anti-cancer agent, or applying an anti-cancer treatment to the subject based on the generated genomic profile. In some embodiments, the called CNAs for the one or more gene loci are used in making suggested treatment decisions for the subject. In some embodiments, the called CNAs for the one or more gene loci are used in applying or administering a treatment to the subject.

Disclosed herein are systems comprising: one or more processors; and a memory communicatively coupled to the one or more processors and configured to store instructions that, when executed by the one or more processors, cause the system to: receive sequence read data for a plurality of sequence reads; estimate a degree of contamination for the sample based on a distribution of allele frequencies (AFs) for a plurality of selected single nucleotide polymorphisms (SNPs) identified within a plurality of gene loci in the sequence read data; segment the sequence read data into two or more segments, wherein each segment has a same copy number, and wherein sequence read data comprising SNPs that exhibit an allele frequency below a first threshold are excluded from the segmenting process; classify a SNP detected on a segment of the two or more segments as aberrant when the SNP exhibits an allele frequency that is different from an allele frequency for other SNPs detected on the same segment; adjust the first threshold based on a distribution of aberrant SNP allele frequencies; repeat the segmenting, classifying, and adjusting steps when the first threshold is increased; and output the segmentation data and a final threshold as the estimated degree of contamination for the sample. In some embodiments, the instructions further comprise causing the system to use the segmentation data and estimated degree of contamination to build a copy number model that predicts a copy number for the one or more gene loci.

Also disclosed herein are non-transitory computer-readable storage media storing one or more programs, the one or more programs comprising instructions, which when executed by one or more processors of a system, cause the system to: receive sequence read data for a plurality of sequence reads; estimate a degree of contamination for the sample based on a distribution of allele frequencies (AFs) for a plurality of selected single nucleotide polymorphisms (SNPs) identified within a plurality of gene loci in the sequence read data; segment the sequence read data into two or more segments, wherein each segment has a same copy number, and wherein sequence read data comprising SNPs that exhibit an allele frequency below a first threshold are excluded from the segmenting process; classify a SNP detected on a segment of the two or more segments as aberrant when the SNP exhibits an allele frequency that is different from an allele frequency for other SNPs detected on the same segment; adjust the first threshold based on a distribution of aberrant SNP allele frequencies; repeat the segmenting, classifying, and adjusting steps when the first threshold is increased; and output the segmentation data and a final threshold as the estimated degree of contamination for the sample. In some embodiments, the instructions further comprise causing the system to use the segmentation data and estimated degree of contamination to build a copy number model that predicts a copy number for the one or more gene loci.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for performing an iterative contamination detection and segmentation process for processing nucleic acid sequence data.

FIG. 2 provides a non-limiting example of process flowchart for determining an initial estimate for sample contamination based on the distribution of minor allele frequencies for a plurality of selected heterozygous SNPs.

FIG. 3 provides a non-limiting example of a process flowchart for performing iterative segmentation of sequence data based on an initial estimate of sample contamination.

FIG. 4 provides a non-limiting example of a process flowchart for performing a review of SNP minor allele frequency data to identify gene loci data that are likely derived from contaminating DNA and that should thus be excluded from copy number analysis.

FIG. 5 depicts an exemplary computing device, in accordance with some instances of the systems described herein.

FIG. 6 depicts an exemplary computer system or computer network, in accordance with some instances of the systems described herein.

FIG. 7 provides a non-limiting example of plots of log 2 coverage ratio data and minor allele frequency data.

### DETAILED DESCRIPTION

Methods and systems for performing iterative contamination detection and segmentation of sequence read data. The methods comprise estimating a degree of contamination for the sample based on a distribution of allele frequencies (e.g., minor allele frequencies) for a selected set of single nucleotide polymorphisms (SNPs) (e.g., heterozygous single nucleotide polymorphisms (SNPs)). Then, using the estimated degree of contamination as an initial value for a first threshold (e.g., a minor allele frequency (MAF) threshold), the sequencing data is iteratively segmented while simultaneously excluding sequencing data from the segmentation process that comprises SNPs having allele frequencies that are below the first threshold. At each iteration, the remaining SNPs are classified as aberrant (i.e., likely due to contamination) if they have an allele frequency that is different from the allele frequencies for other SNPs detected on the same segment, and the first threshold is incrementally adjusted based a comparison of the distribution of aberrant SNP allele frequencies to the expected distribution of allele frequencies for the selected set of, e.g., heterozygous SNPs. The segmenting, classifying, and first threshold adjusting steps are repeated each time the first threshold is increased. When no further increase of the first threshold is required (or there is no further change in the distribution of aberrant SNP minor allele frequencies, or a specified maximum number of iterations has been reach), the segmentation data and an estimated degree of contamination for the sample (equal to the final value of the first threshold) is output. In some instances, the method further comprises using the segmentation data and estimated degree of contamination to build a copy number model that predicts a copy number for the one or more gene loci.

In some instances, for example, the disclosed methods for detecting contamination in sequence read data for a sample comprise: receiving, at one or more processors, sequence read data for a plurality of sequence reads; estimating a degree of contamination for the sample based on a distribution of allele frequencies (AFs) for a plurality of selected single nucleotide polymorphisms (SNPs) identified within a plurality of gene loci in the sequence read data; segmenting the sequence read data into two or more segments, wherein each segment has a same copy number, and wherein sequence read data comprising SNPs that exhibit an allele frequency below a first threshold are excluded from the segmenting process; classifying a SNP detected on a segment of the two or more segments as aberrant when the SNP exhibits an allele frequency that is different from an allele frequency for other SNPs detected on the same segment; adjusting the first threshold based on a distribution of aberrant SNP allele frequencies; repeating the segmenting, classifying, and adjusting steps when the first threshold is increased; and outputting the segmentation data and a final threshold as the estimated degree of contamination for the sample. In some instances, the method may further comprise using the segmentation data and estimated degree of contamination output by the one or more processors to build a copy number model that predicts a copy number for the one or more gene loci.

The disclosed methods and systems reduce or eliminate the erroneous detection and calling of variant sequences that are not actually present in the patient sample, enable more accurate copy number modeling of sequence read data, and thereby result in more reliable detection and calling of copy number alterations in one or more gene loci represented by the sequence data for a patient sample.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “about” a number or value refers to that number or value plus or minus 10% of that number or value. The term ‘about’ when used in the context of a range refers to that range minus 10% of its lowest value and plus 10% of its greatest value.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence.

In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “major allele” refers to the most common allele for a given gene locus or single nucleotide polymorphism (SNP).

As used herein, the term “minor allele” refers to the less common allele for a given gene locus or SNP. The minor allele is the second most common allele for genomic loci (e.g., gene loci, SNP loci, etc.) where more than two alleles are observed.

As used herein, the terms “biallelic gene locus” and “biallelic SNP” refer to a gene locos or SNP, respectively, that contains two observed alleles, counting the reference as one. A biallelic gene locus or biallelic SNP may thus contain two observed alleles, a reference allele (i.e., an allele matching the allele present in a reference genome, such as GRCh38) and an alternate allele.

As used herein, the term “segmentation” (or “sequence segmentation”) refers to a process for partitioning of the sequence read data into a number of non-overlapping segments that cover all sequence read data points, such that each segment of the plurality of segments is as homogeneous as possible and all sequence reads associated with a given segment have the same copy number. In some instances, segmentation may be performed by processing aligned sequence read data (or other sequencing-related data, e.g., coverage data, allele frequency data, etc., derived from the sequence read data) using any of a variety of methods known to those of skill in the art (see., e.g., Braun and Miller (1998), “Statistical methods for DNA sequence segmentation”, Statistical Science 13(2): 142-162). Examples of segmentation methods include, but are not limited to, circular binary segmentation (CBS) methods, maximum likelihood methods, hidden Markov chain methods, walking Markov methods, Bayesian methods, long-range correlation methods, change point methods, or any combination thereof.

As used herein, the term “ploidy” refers to the average copy number for a plurality of gene loci in a tumor sample. In some instances, the “ploidy” of a tumor sample may differ from the number of complete sets of chromosomes in a cell, and hence the number of possible alleles for autosomal genes (i.e., genes located on numbered, non-sex chromosomes), due to the heterogeneity of the tumor sample (i.e., the variation in tumor sample purity).

The section headings used herein are for organizational purposes only and are not to be construed as limiting the subject matter described.

### Methods for Performing Iterative Contamination Detection and Segmentation

The disclosed methods for performing iterative contamination detection and segmentation address two primary objectives: (i) to detect and estimate the degree of contamination in a sequencing sample, and (ii) to exclude the contamination as a source of error in downstream copy number modeling. The ability to detect contamination in a sample, to estimate the degree to which the sample is contaminated, and to remove the contaminating sequence read data allows one to, for example, identify samples with significant contamination that must therefore be failed by a variant calling or copy number calling pipeline for processing nucleic acid sequence data (transplant cases can be an exception to this case; in transplant cases, the “contaminant” is known and thus variants can still be reported). Additionally, the ability to remove contamination as a source of error in downstream variant calling or copy number modeling allows one to minimize or eliminate erroneous variant calls and to more accurately detect and call copy number alterations (CNAs). Left uncorrected, sequence read data for contaminated samples can look like very like data for high purity (i.e., high tumor fraction) samples due to the presence low-frequency SNPs.

When two human nucleic acid samples are mixed, the allele frequency (AF) profiles for common SNPs are substantially affected. Table 1 describes the impact of contamination for a single SNP in the case of low-level contamination:

TABLE 1

| Sample 1 | Sample 2 | |
|---|---|---|
| Genotype | Genotype | Sample 1 / Sample 2 Mixture |
| A/A | A/A | AA at expected allele frequencies |
| A/A | A/B or B/B | A/B present, but with unusually low |
| | | allele frequency for B allele |
| A/B | A/A, A/B, or B/B | A/B present with slightly shifted |
| | | allele frequency. |
| B/B | A/A or A/B | A/B present, but with unusually low |
| | | frequency for A allele |
| B/B | B/B | B/B at expected allele frequencies |

There are several strategies that can be used to detect sample contamination. In one approach, for example, one can look for enrichment of low allele frequency SNPs. Low level contamination usually generates a distinct band of low-minor-allele-frequency SNPs. However, this approach can be confounded by samples that have low allele frequency SNPs due to tumor aneuploidy. The most problematic situation is in the case of high-purity (high tumor fraction), genome-wide loss of heterozygosity, where the tumor has lost one copy of each chromosome and all SNPs appear at low allele frequency.

A second strategy for detecting sample contamination is based on looking for excess heterozygosity. Population-wide, SNPs are generally found in Hardy-Weinberg equilibrium. This principle, when applied to a set of SNPs in a given sample (particularly when applied to a set of very common, biallelic SNPs), prescribes a particular distribution of genotypes. Particularly, it sets a constraint on what level of heterozygosity can reasonably be observed by chance. Contamination of a sample leads to an excess apparent heterozygosity, which can be an effective means of detecting contamination. This approach avoids issues related to sample purity (tumor fraction), but can be confounded by ancestry (including variation in overall heterozygosity across populations) and the difficulty in determining a set of uniformly polymorphic SNPs to use for the test.

A third strategy comprising looking for SNPs with inconsistent minor allele frequencies relative to their neighbors, and forms the basis for the methods described herein.

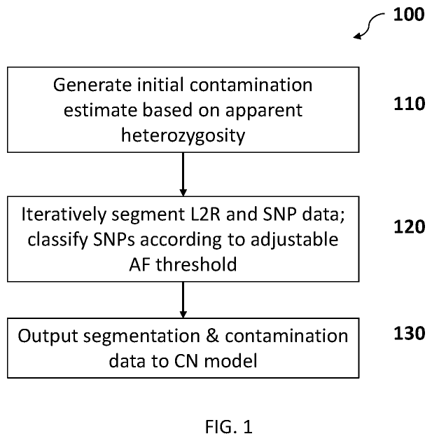

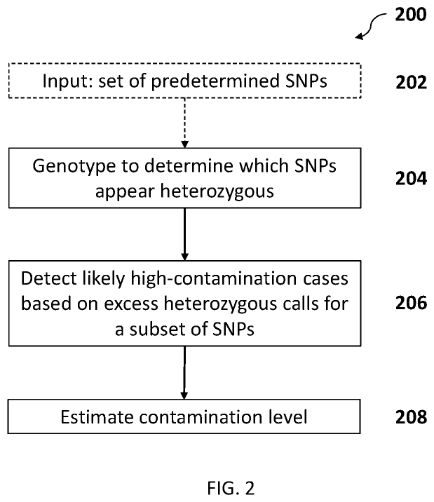

FIG. 1 provides a non-limiting example of a process flowchart for performing an iterative contamination detection and segmentation process 100 for processing nucleic acid sequence data. In step 110, an initial estimate of the degree of contamination in a sample is made based on determining the apparent heterozygosity of the sample using a plurality of selected heterozygous SNPs identified in sequence read data for a plurality of sequence reads overlapping one or more gene loci within one or more subgenomic intervals. The process for generating the initial estimate of contamination will be described in more detail below with respect to FIG. 2.

In some instances, the sequence read data may be converted to coverage ratio data (or to log 2 coverage ratio (L2R) data) prior to further processing. In some instances, the coverage ratio data for the sample (e.g., a patient tumor sample) is determined by aligning a plurality of sequence reads that overlap one or more gene loci within one or more subgenomic intervals in the sample and in a control (e.g., a paired normal control, a process-matched control, or a “panel of normal” control) to a reference genome (e.g., the GRCh38 human reference genome), and determining a number of sequence reads that overlap each of the one or more gene loci within the one or more subgenomic intervals in the sample and in the control in order to normalize the coverage for the tumor sample to that in the control. In some instances, e.g., if a paired normal control sample is not available, a process-matched control (e.g., a mixture of DNA from a plurality of HapMap cell lines) may be used instead of the paired normal control to normalize coverage. In some instances, e.g., if a paired normal control sample is not available, a “panel of normal” control may be used instead of the paired normal control to normalize coverage.

One method of applying a “panel of normal” or “Tangent normalization” control method to normalize sequencing coverage is described by Tabak, et al. (2019) “The Tangent copy-number inference pipeline for cancer genome analyses”, https://www.biorxiv.org/content/10.1101/566505v1.full.pdf. The Tangent normalization method is a method of normalizing tumor data in order to deal with noise in the data. Specifically, the Tangent method deals with reducing systemic noise resulting from differences in the experimental conditions under which sequencing data from tumors and/or their normal controls were generated. It has been shown that the Tangent normalization method yields a greater reduction in noise than conventional normalization methods.

In some instances, the allele fraction data for the sample (e.g., a patient tumor sample) is determined by aligning a plurality of sequence reads that overlap one or more gene loci within one or more subgenomic intervals in the sample to a reference genome (e.g., the GRCh38 human reference genome), detecting a number of different alleles present at the one or more gene loci in the one or more subgenomic intervals in the sample, and determining an allele fraction for the different alleles present at the one or more gene loci by dividing the number of sequence reads identified for a given allele sequence by the total number of sequence reads identified for the gene locus.

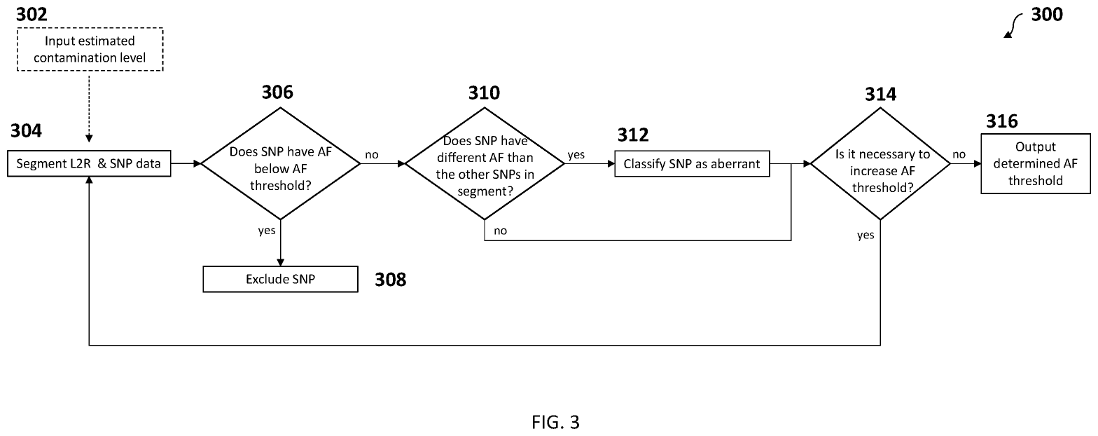

At step 120 in FIG. 1, an iterative process of contamination detection and segmentation of the sequence read data is performed. As noted above, the sequence read data for a plurality of sequence reads that overlap one or more gene loci in one or more subgenomic intervals in the sample and in a control may be aligned to a reference genome, and a number of sequence reads that overlap each of the one or more gene loci within the one or more subgenomic intervals in the sample and in the control may be determined in order to normalize the coverage for the tumor sample to that in the control (i.e., to determine a coverage ratio). In some instances, the coverage ratio data may be further transformed into L2R data. An iterative process is then performed using the L2R data for the one or more gene loci (and for associated SNPs) to adjust an allele frequency (AF) threshold (e.g., a minor allele frequency (MAF) threshold) used to detect likely contamination, remove the associated coverage or L2R data from further analysis, and perform segmentation of the coverage or L2R data. The process for iteratively detecting likely contamination, removing the associated coverage or L2R data from further analysis, and performing segmentation will be described in more detail below with respect to FIG. 3.

At step 130 in FIG. 1, the segmentation and contamination data determined using the iterative process in step 120 is output. In some instances, the segmentation and contamination data output at step 130 is used, for example, as input to a copy number model that best accounts for the coverage ratio and allele fraction data associated with the plurality of sequence reads for the one or more gene loci.

FIG. 2 provides a non-limiting example of a flowchart for a process 200 used to determine an initial estimate for sample contamination based on the distribution of allele frequencies (e.g., minor allele frequencies) for a plurality of selected SNPs (e.g., a plurality of selected heterozygous SNPs) associated with the one or more gene loci. A predetermined set of SNPs are input at step 202, and genotyped at step 204 to identify a subset of SNPs that appear heterozygous.

For the initial estimate of contamination, typically only a small number of SNP loci (e.g., on the order of 1,000) are considered. In some instances, the plurality of selected heterozygous single nucleotide polymorphisms (SNPs) comprises biallelic SNPs having unbiased heterozygous allele frequencies of about 50%. In some instances, the plurality of selected heterozygous single nucleotide polymorphisms (SNPs) comprises common biallelic SNPs having reference and alternate alleles that are observed at greater than, e.g., 20% global MAF (i.e., observed at greater than, e.g., 20% in a default global population as reported in the Single Nucleotide Polymorphism Database (dbSNP) or in the Genome Aggregation Database (GnomAD)).

In some instances the number of selected heterozygous SNP loci used to determine an initial estimate of contamination may range from about 100 to about 1,000,000 SNP loci. In some instances, the number of selected heterozygous SNP loci may be at least 100, at least 1,000, at least 10,000, at least 100,000, or at least 1,000,000. In some instances, the number of selected heterozygous SNP loci may be at most 1,000,000, at most 100,000, at most 10,000, at most 1,000, or at most 100. Any of the lower and upper values described in this paragraph may be combined to form a range included within the present disclosure, for example, in some instances, the number of selected heterozygous SNP loci may range from 1,000 to 10,000. Those of skill in the art will recognize that the number of selected heterozygous SNP loci may have any value within this range, e.g., about 1,012 SNP loci.

In some instances, the selected heterozygous SNP loci may comprise biallelic SNPs having reference and alternate allele frequencies of at least 10%, at least 15%, at least 20%, at least 25%, at least 30%, at least 35%, at least 40%, or at least 45% global MAF.

At step 206 in FIG. 2, coverage ratio or L2R data likely to be associated with contamination is detected based on an excess number of heterozygous calls for the selected SNPs in the sample (e.g., identification of a subset of selected heterozygous SNPs having inconsistent minor allele frequencies relative to their neighboring target gene loci, SNP loci, or introns). Thus, an initial estimate of the degree of contamination for the sample is output at step 208 based on the distribution of allele frequencies for the plurality of selected heterozygous SNPs, and comprises determining a percentage of the selected heterozygous SNPs that have AFs (e.g., MAFs) that are significantly different than an expected AF distribution (e.g., an expected MAF distribution) for the plurality of selected heterozygous SNPs identified within the plurality of gene loci. In some instances, determining the percentage of the selected heterozygous SNPs that have AFs (e.g., MAFs) that are significantly different than the expected AF distribution (e.g., an expected MAF distribution) for the plurality of selected heterozygous SNPs may comprise determining the percentage of the selected heterozygous SNPs that have AFs that differ from the expected AF distribution for the plurality of selected heterozygous SNPs by at least a second threshold. In some instances, the second threshold may be at least 1, at least 2, at least 3, or at least 4 standard deviations from the mean of the expected allele frequency distribution for the plurality of selected heterozygous SNPs.

FIG. 3 provides a non-limiting example of a flowchart for a process 300 for performing iterative segmentation of sequence data based on an initial estimate of sample contamination. An initial estimate of sample contamination level (as determined by process 200 illustrated in FIG. 2) is input at step 302 and used as the initial value for an adjustable first threshold (e.g., an adjustable AF threshold or MAF threshold). The iterative segmentation process is initiated at step 304 using the L2R data for one or more gene loci and associated heterozygous SNPs. At step 306, the allele frequency for each of the predetermined set of SNPs is compared to the current AF threshold (e.g., MAF threshold) (i.e., to identify L2R and allele frequency data that is likely due to contamination), and excluded from further analysis (i.e., excluded from the data set used for segmentation and copy number modeling) at step 308 if it has an allele frequency that is below the current AF threshold (e.g., MAF threshold).

In some instances, the first threshold (e.g., an allele frequency threshold, or minor allele frequency (MAF) threshold) may range from about 0.1 to about 0.9 (in fractional units). In some instances, the first threshold may be at least 0.1, at least 0.2, at least 0.3, at least 0.4, at least 0.5, at least 0.6, at least 0.7, at least 0.8, or at least 0.9. In some instances, the first threshold may be at most 0.9, at most 0.8, at most 0.7, at most 0.6, at most 0.5, at most 0.4, at most 0.3, at most 0.2, or at most 0.1.

In some instances, the first threshold (e.g., an allele frequency threshold, or minor allele frequency (MAF) threshold) may range from about 10% to about 90% (in percentage units). In some instances, the first threshold may be at least 10%, at least 20%, at least 30%, at least 40%, at least 50%, at least 60%, at least 70%, at least 80%, or at least 90%. In some instances, the first threshold may be at most 90%, at most 80%, at most 70%, at most 60%, at most 50%, at most 40%, at most 30%, at most 20%, or at most 10%.

If the SNP allele frequency data is determined to be above the current AF threshold (e.g., MAF threshold) at step 306, a comparison to the allele frequencies for other SNPs on the same segment is made at step 310. In some instances, a SNP is classified as aberrant at step 312 if it exhibits an allele frequency that is different from the allele frequencies for other SNPs detected on the same segment based on an absolute value of the difference in allele frequency. In some instances, a SNP is classified as aberrant at step 312 if it exhibits an allele frequency that is different from the allele frequency for other SNPS detected on the same segment based on a statistical analysis, for example, a t-test.

At step 314 in FIG. 3, a determination is made as to whether or not the current AF threshold (e.g., MAF threshold) should be increased. The AF threshold may be increased iteratively in incremental steps based on the overall distribution of aberrant SNP minor allele frequencies. In some instances, the AF threshold is incrementally adjusted to reduce the number of SNPs classified as aberrant, and where the AF threshold is set based on a percentage of the heterozygous SNPs that have AFs that are significantly different than an expected AF distribution for the selected (predetermined) set of heterozygous SNPs identified within the one or more gene loci. For true contamination, one would expect there to be a sizeable number of contaminating SNPs (e.g., thousands if they are all at detectable levels), so one doesn't need to take the highest allele frequency observed to determine an AF threshold (e.g., a MAF threshold). Instead, one can look to a position in the distribution, e.g., the 50th highest allele frequency (for example, corresponding to a particular percentile of the distribution expected due to contamination). The AF threshold is then adjusted based on a number of different criteria to account for variations in data quality (e.g., the difference in observed SNP allele frequencies, the highest allele frequency observed for the sample, the case where all SNPs are classified as aberrant, etc.). In some instances, the AF threshold is incrementally adjusted based on a percentage of SNPs identified in the sample that have allele frequencies that differ from an expected allele frequency distribution for the plurality of selected heterozygous SNPs by at least a third threshold. In some instances, the third threshold is at least 1, at least 2, at least 3, or at least 4 standard deviations from the mean of the expected allele frequency distribution for the plurality of selected heterozygous SNPs.

If it is necessary to increase the AF threshold at step 314, the iterative segmentation process is repeated by looping back to step 304. In some instances, the segmentation is performed using a circular binary segmentation (CBS) method, a maximum likelihood method, a hidden Markov chain method, a walking Markov method, a Bayesian method, a long-range correlation method, or a change point method. In some instances, the segmentation is performed using a change point method, and the change point method is a pruned exact linear time (PELT) method. In some instances, the segmentation loop depicted in FIG. 3 (steps 304 to 314) may be repeated at least 1, 2, 3, 4, 5, 6, 7, 8, 9, 10, or more than 10 times.

If there is no need to increase the AF threshold at step 314, the current value of the AF threshold is output at step 316 as the final estimate of the degree of contamination in the sample.

In some instances, depending on the quality of the sequencing data, the limit of detection for detecting contamination in the sample using the disclosed methods is less than about 10%, less than about 5%, less than about 4%, less than about 3%, less than about 2%, less than about 1%, less than about 0.5%, or less than about 0.1%.

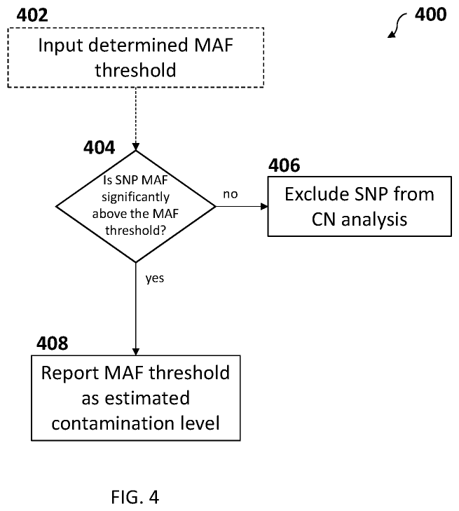

FIG. 4 provides a non-limiting example of a flowchart for a process 400 used to perform a review and filtering of SNP minor allele frequency data to identify gene loci data that are likely derived from contaminating DNA and that should thus be excluded from copy number analysis. The final value of the AF threshold (e.g., MAF threshold) determined by process 300 depicted in FIG. 3 is input at step 402. At step 404, the minor allele frequency for each SNP in the predetermined (selected) set of heterozygous SNPs is compared to the final value of the AF threshold. SNPs having an AF that is not significantly above the AF threshold are excluded (along with L2R and allele frequency data for gene loci on the same segment as the SNP) from use in copy number modeling. SNPs having an AF that is significantly above the AF threshold are included in copy number modeling (along with L2R and allele frequency data for gene loci on the same segment as the SNP), and the final value of the AF threshold is reported as the estimated degree of contamination in the sample.

In some instances, the disclosed methods for performing iterative contamination detection and segmentation may be applied to sequence read data covering a panel of gene loci comprising at least 10, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 320, at least 340, at least 360, at least 380, at least 400, or more than 400 gene loci. In some instances, the panel may further comprise a plurality of genome-wide SNP loci, e.g., comprising at least 100, at least 200, at least 300, at least 400, at least 500, at least 600, at least 700, at least 800, at least 900, at least 1,000, at least 2,000, at least 3,000, at least 4,000, at least 5,000, at least 600, at least 7,000, at least 8,000, at least 9,000, or at least 10,000 SNP loci. In some instances, the panel may comprise at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 5,500, at least 6,000, at least 6,500, at least 7,000, at least 7,500, at least 8,000, at least 8,500, at least 9,000, at least 9,500, at least 10,000, at least 11,000, at least 12,000, at least 13,000, at least 14,000, or at least 15,000 target loci comprising a combination of gene loci, SNP loci, exon loci, intron loci, or any combination thereof.

In some instances, the predetermined set (or selected subset) of heterozygous SNP loci may comprise at least 100, at least 500, at least 1,000, at least 5,000, at least 10,000, at least 50,000, at least 100,000, at least 500,000, or at least 1,000,000 SNP loci.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for performing iterative contamination detection and segmentation may be used as part of a copy number alteration calling pipeline that, in turn, may be used to diagnose the presence of disease (e.g., cancer, genetic disorders (such as Down Syndrome and Fragile X), neurological disorders, or any other disease type where copy number is relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for performing iterative contamination detection and segmentation may be used as part of a copy number alteration calling pipeline that, in turn, may be used to predict genetic disorders in fetal DNA. (e.g., for invasive or non-invasive prenatal testing). For example, sequence read data obtained sequencing fetal DNA extracted from samples obtained using invasive amniocentesis, chorionic villus sampling (CVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify copy number alterations associated with, e.g., Down Syndrome (trisomy 21), trisomy 18, trisomy 13, and extra or missing copies of the X and Y chromosomes.

In some instances, the disclosed methods for performing iterative contamination detection and segmentation may be used as part of a copy number alteration calling pipeline that, in turn, may be used to select a subject (e.g., a patient) for a clinical trial based on the CNA value determined for one or more gene loci. In some instances, patient selection for clinical trials based on, e.g., identification of CNAs at one or more gene loci, may accelerate the development of targeted therapies and improve the healthcare outcomes for treatment decisions.

In some instances, the disclosed methods for performing iterative contamination detection and segmentation may be used as part of a copy number alteration calling pipeline that, in turn, may be used to select an appropriate therapy or treatment (e.g., a cancer therapy or cancer treatment) for a subject. In some instances, for example, the cancer therapy or treatment may comprise use of a poly(ADP-ribose) polymerase inhibitor (PARPi), a platinum compound, chemotherapy, radiation therapy, a targeted therapy (e.g., immunotherapy), surgery, or any combination thereof.

In some instances, the disclosed methods for performing iterative contamination detection and segmentation may be used as part of a copy number alteration calling pipeline that, in turn, may be used in treating a disease (e.g., a cancer) in a subject. For example, in response to calling CNAs using any of the methods disclosed herein, an effective amount of a cancer therapy or cancer treatment may be administered to the subject.

In some instances, the disclosed methods for performing iterative contamination detection and segmentation may be used as part of a copy number alteration calling pipeline that, in turn, may be used for monitoring disease progression or recurrence (e.g., cancer or tumor progression or recurrence) in a subject. For example, in some instances, the methods may be used to call CNAs in a first sample obtained from the subject at a first time point, and used to call CNAs in a second sample obtained from the subject at a second time point, where comparison of the first determination of CNAs and the second determination of CNAs allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., a cancer treatment or cancer therapy) for a subject, e.g., by adjusting a treatment dose and/or selecting a different treatment in response to a change in the determination of one or more CNAs using a copy number alteration calling pipeline that incorporates the iterative contamination detection and segmentation methods disclosed herein.

In some instances, the detection of copy number alterations (CNAs) using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for performing iterative contamination detection and segmentation as part of a copy number alteration calling pipeline may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for performing iterative contamination detection and segmentation and calling CNAs as part of a genomic profiling process (or inclusion of the output from the disclosed methods for performing iterative contamination detection and segmentation and calling CNAs as part of the genomic profile of the subject) can improve the validity of, e.g., disease detection calls and treatment decisions, made on the basis of the genomic profile by, for example, independently confirming the presence of CNAs in one or more gene loci in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with a cancer therapy (or cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with a cancer therapy (or cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion Recover All™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9): 1297-303; Ye, K., et al., Bioinformatics, 2009; 25(21): 2865-71; Lunter, G., and Goodson, M., Genome Res. 2011; 21(6): 936-9; and Li, H., et al. (2009), Bioinformatics 25(16): 2078-9.

Methods for generating indel calls and individual-level genotype likelihoods include, e.g., the Dindel algorithm (Albers, C. A., et al., Genome Res. 2011; 21(6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for performing iterative contamination detection and segmentation (e.g., as standalone procedures, or as part of a copy number alteration calling pipeline) in a sample from a subject. The systems may comprise, e.g., one or more processors, and a memory unit communicatively coupled to the one or more processors and configured to store instructions that, when executed by the one or more processors, cause the system to: receive sequence read data for a plurality of sequence reads; estimate a degree of contamination for the sample based on a distribution of allele frequencies (AFs) for a plurality of selected single nucleotide polymorphisms (SNPs) identified within a plurality of gene loci in the sequence read data; segment the sequence read data into two or more segments, wherein each segment has a same copy number, and wherein sequence read data comprising SNPs that exhibit an allele frequency below a first threshold are excluded from the segmenting process; classify a SNP detected on a segment of the two or more segments as aberrant when the SNP exhibits an allele frequency that is different from an allele frequency for other SNPs detected on the same segment; adjust the first threshold based on a distribution of aberrant SNP allele frequencies; repeat the segmenting, classifying, and adjusting steps when the first threshold is increased; and output the segmentation data and a final threshold as the estimated degree of contamination for the sample.

In some instances, the disclosed systems may further comprise a sequencer, e.g., a next generation sequencer (also referred to as a massively parallel sequencer). Examples of next generation (or massively parallel) sequencing platforms include, but are not limited to, the Roche 454, Illumina Solexa, ABI-SOLID, ION Torrent, or Pacific Bioscience sequencing platforms.

In some instances, the disclosed systems may be used for performing iterative contamination detection and segmentation (and/or for copy number alteration calling) in any of a variety of samples as described herein (e.g., a tissue sample, biopsy sample, hematological sample, or liquid biopsy sample derived from the subject).

In some instances, the plurality of gene loci for which sequencing data is processed to determine a degree of contamination and/or to call CNAs may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of copy number alterations in one or more gene loci is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

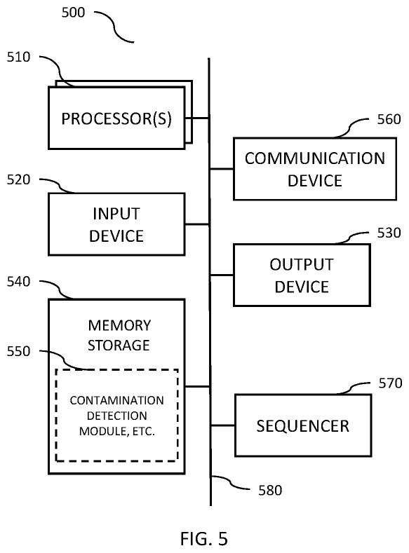

FIG. 5 illustrates an example of a computing device or system in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more processor(s) 510, input devices 520, output devices 530, memory or storage devices 540, communication devices 560, and nucleic acid sequencers 570. Software 550 residing in memory or storage device 540 may comprise, e.g., an operating system as well as software for executing the methods described herein. Input device 520 and output device 530 can generally correspond to those described herein, and can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage (e.g., an electrical, magnetic or optical memory including a RAM (volatile and non-volatile), cache, hard drive, or removable storage disk). Communication device 560 can include any suitable device capable of transmitting and receiving signals over a network, such as a network interface chip or device. The components of the computer can be connected in any suitable manner, such as via a wired media (e.g., a physical system bus 580, Ethernet connection, or any other wire transfer technology) or wirelessly (e.g., Bluetooth®, Wi-Fi®, or any other wireless technology).

Software module 550, which can be stored as executable instructions in storage 540 and executed by processor(s) 510, can include, for example, an operating system and/or the processes that embody the functionality of the methods of the present disclosure (e.g., as embodied in the devices as described herein).

Software module 550 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 540, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

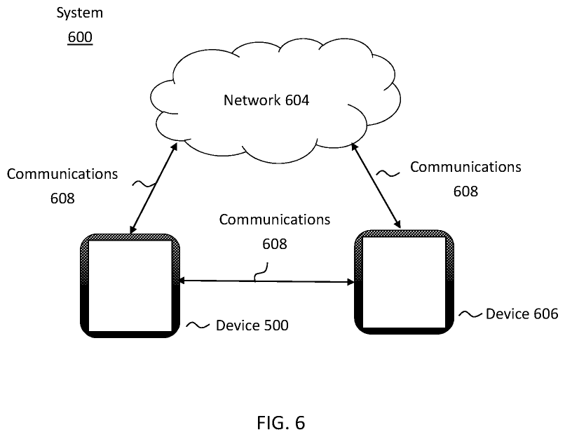

Device 500 may be connected to a network (e.g., network 604, as shown in FIG. 6 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can be implemented using any operating system, e.g., an operating system suitable for operating on the network. Software module 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 510.

Device 500 can further include a sequencer 570, which can be any suitable nucleic acid sequencing instrument.

FIG. 6 illustrates an example of a computing system in accordance with one embodiment. In system 600, device 500 (e.g., as described above and illustrated in FIG. 5) is connected to network 604, which is also connected to device 606. In some embodiments, device 606 is a sequencer.

Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system.

Devices 500 and 606 may communicate, e.g., using suitable communication interfaces via network 604, such as a Local Area Network (LAN), Virtual Private Network (VPN), or the Internet. In some embodiments, network 604 can be, for example, the Internet, an intranet, a virtual private network, a cloud network, a wired network, or a wireless network. Devices 500 and 606 may communicate, in part or in whole, via wireless or hardwired communications, such as Ethernet, IEEE 802.11b wireless, or the like. Additionally, devices 500 and 606 may communicate, e.g., using suitable communication interfaces, via a second network, such as a mobile/cellular network. Communication between devices 500 and 606 may further include or communicate with various servers such as a mail server, mobile server, media server, telephone server, and the like. In some embodiments, Devices 500 and 606 can communicate directly (instead of, or in addition to, communicating via network 604), e.g., via wireless or hardwired communications, such as Ethernet, IEEE 802.11b wireless, or the like. In some embodiments, devices 500 and 606 communicate via communications 608, which can be a direct connection or can occur via a network (e.g., network 604).

One or all of devices 500 and 606 generally include logic (e.g., http web server logic) or are programmed to format data, accessed from local or remote databases or other sources of data and content, for providing and/or receiving information via network 604 according to various examples described herein.

### EXAMPLES

### Example 1—Exemplary Log 2 Coverage Ratio Data

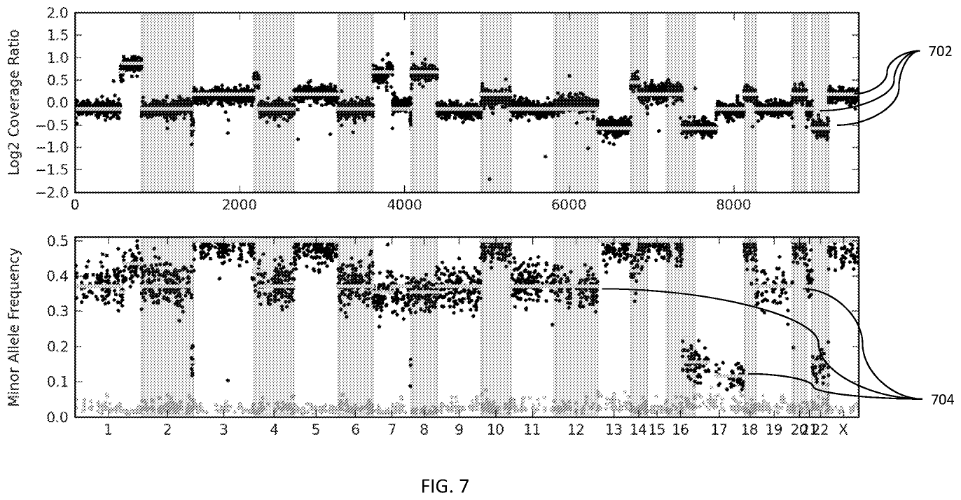

FIG. 7 provides a non-limiting example of plots of log 2 coverage ratio (L2R) data (upper panel) and minor allele frequency (MAF) data (lower panel) generated using the disclosed methods for iterative contamination detection and segmentation. The minor allele frequency data points for aberrant SNPs are colored in orange in the lower panel, and have been excluded from the copy number analysis for this sample. The contamination estimate generated using the disclosed methods was 4.6%. The horizontal bars 702 and 704 correspond to the expected levels for the L2R and MAF data respectively, given the best-fit mode for the copy number model.

### EXEMPLARY IMPLEMENTATIONS

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules, wherein one or more of the plurality of sequencing reads overlap one or more gene loci within one or more subgenomic intervals in the sample;
   receiving, at one or more processors, sequence read data for the plurality of sequence reads;
   estimating, using the one or more processors, a degree of contamination for the sample based on a predetermined distribution of allele frequencies (AFs) for a plurality of selected single nucleotide polymorphisms (SNPs) identified within a plurality of gene loci in the sequence read data;
   segmenting, using the one or more processors, the sequence read data into two or more segments, wherein each segment has a same copy number, and wherein sequence read data comprising SNPs that exhibit an allele frequency below a first threshold are excluded from the segmenting process;
   classifying, using the one or more processors, a SNP detected on a segment of the two or more segments as aberrant when the SNP exhibits an allele frequency that is different from an allele frequency for other SNPs detected on the same segment;
   adjusting, using the one or more processors, the first threshold based on a distribution of aberrant SNP allele frequencies;
   repeating the segmenting, classifying, and adjusting steps when the first threshold is increased; and
   outputting, using the one or more processors, the segmentation data and a final threshold as the estimated degree of contamination for the sample.
2. The method of clause 1, further comprising setting an initial value for the first threshold as equal to the estimated degree of contamination for the sample.
3. The method of clause 1 or clause 2, wherein the plurality of selected single nucleotide polymorphisms (SNPs) comprises a plurality of selected heterozygous single nucleotide polymorphisms (SNPs).
4. The method of any one of clauses 1 to 3, wherein the predetermined distribution of allele frequencies (AFs) for the plurality of selected single nucleotide polymorphisms (SNPs) comprises a predetermined distribution of minor allele frequencies (MAFs) for the plurality of selected single nucleotide polymorphisms (SNPs).
5. The method of any one of clauses 1 to 4, further comprising using the segmentation data and estimated degree of contamination output by the one or more processors to build a copy number model that predicts a copy number for the one or more gene loci.
6. The method of any one of clauses 1 to 5, further comprising excluding all sequence read data for gene loci on a same segment as SNPs that exhibit an allele frequency below the final threshold from a copy number analysis for the one or more gene loci.
7. The method of any one of clauses 1 to 6, wherein estimating the degree of contamination for the sample based on the distribution of minor allele frequencies for the plurality of selected SNPs comprises determining a percentage of the SNPs identified in the sample that have allele frequencies that differ from an expected allele frequency distribution for a plurality of selected heterozygous SNPS identified within the plurality of gene loci by at least a second threshold.
8. The method of any one of clauses 1 to 7, wherein a SNP is classified as aberrant when the SNP exhibits an allele frequency that is different from the allele frequency for other SNPs detected on the same segment based on an absolute value of the difference in allele frequency.
9. The method of any one of clauses 1 to 8, wherein a SNP is classified as aberrant when the SNP exhibits an allele frequency that is different from the allele frequency for other SNPS detected on the same segment based on a statistical analysis.
10. The method of any one of clauses 1 to 9, wherein the segmenting step is performed using a circular binary segmentation (CBS) method, a maximum likelihood method, a hidden Markov chain method, a walking Markov method, a Bayesian method, a long-range correlation method, or a change point method.
11. The method of clause 10, wherein the segmenting is performed using a change point method, and the change point method is a pruned exact linear time (PELT) method.
12. The method of any one of clauses 1 to 11, wherein the first threshold is incrementally adjusted to reduce a number of SNPs classified as aberrant, and wherein the first threshold is set based on a percentage of SNPs identified in the sample that have allele frequencies that differ from an expected allele frequency distribution for a plurality of selected heterozygous SNPS identified within the plurality of gene loci by at least a third threshold.
13. The method of any one of clauses 1 to 12, wherein the subject is suspected of having or is determined to have a disease.
14. The method of clause 13, wherein the disease is cancer.
15. The method of any one of clauses 1 to 14, wherein the method is used as part of a copy number alteration (CNA) calling pipeline for routine testing.
16. The method of any one of clauses 1 to 15, wherein the method is used as part of a copy number alteration (CNA) calling pipeline for prenatal testing.
17. The method of any one of clauses 1 to 16, further comprising collecting the sample from the subject.
18. The method of any one of clauses 1 to 17, wherein the sample comprises a tissue biopsy sample, a liquid biopsy sample, or a normal control.
19. The method of clause 18, wherein the sample is a tissue biopsy sample and comprises bone marrow.
20. The method of clause 18, wherein the sample is a liquid biopsy sample and comprises blood, plasma, cerebrospinal fluid, sputum, stool, urine, or saliva.
21. The method of clause 18, wherein the sample is a liquid biopsy sample and comprises circulating tumor cells (CTCs).
22. The method of clause 18, wherein the sample is a liquid biopsy sample and comprises cell-free DNA (cfDNA), circulating tumor DNA (ctDNA), or any combination thereof.
23. The method of any one of clauses 1 to 22, wherein the plurality of nucleic acid molecules comprises a mixture of tumor nucleic acid molecules and non-tumor nucleic acid molecules.
24. The method of clause 23, wherein the tumor nucleic acid molecules are derived from a tumor portion of a heterogeneous tissue biopsy sample, and the non-tumor nucleic acid molecules are derived from a normal portion of the heterogeneous tissue biopsy sample.
25. The method of clause 23, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
26. The method of any one of clauses 1 to 25, wherein the one or more adapters comprise amplification primers, flow cell adaptor sequences, substrate adapter sequences, or sample index sequences.
27. The method of any one of clauses 1 to 26, wherein the captured nucleic acid molecules are captured from the amplified nucleic acid molecules by hybridization to one or more bait molecules.
28. The method of clause 27, wherein the one or more bait molecules comprise one or more nucleic acid molecules, each comprising a region that is complementary to a region of a captured nucleic acid molecule.
29. The method of any one of clauses 1 to 28, wherein amplifying nucleic acid molecules comprises performing a polymerase chain reaction (PCR) amplification technique, a non-PCR amplification technique, or an isothermal amplification technique.
30. The method of any one of clauses 1 to 29, wherein the sequencing comprises use of a massively parallel sequencing (MPS) technique, whole genome sequencing (WGS), whole exome sequencing, targeted sequencing, direct sequencing, or Sanger sequencing technique.
31. The method of clause 30, wherein the sequencing comprises massively parallel sequencing, and the massively parallel sequencing technique comprises next generation sequencing (NGS).
32. The method of clause 31, wherein the next generation sequencing (NGS) comprises paired end sequencing.
33. The method of any one of clauses 1 to 32, wherein the sequencer comprises a next generation sequencer.
34. The method of any one of clauses 5 to 33, further comprising generating, by the one or more processors, a report indicating the predicted copy number for the one or more gene loci.
35. The method of clause 34, further comprising transmitting the report to a healthcare provider.
36. The method of clause 35, wherein the report is transmitted via a computer network or a peer-to-peer connection.
37. A method for detecting contamination in sequence read data for a sample from a subject, the method comprising:
   receiving, at one or more processors, sequence read data for a plurality of sequence reads;
   estimating, using the one or more processors, a degree of contamination for the sample based on a predetermined distribution of allele frequencies (AFs) for a plurality of selected single nucleotide polymorphisms (SNPs) identified within a plurality of gene loci in the sequence read data;
   segmenting, using the one or more processors, the sequence read data into two or more segments, wherein each segment has a same copy number, and wherein sequence read data comprising SNPs that exhibit an allele frequency below a first threshold are excluded from the segmenting process;
   classifying, using the one or more processors, a SNP detected on a segment of the two or more segments as aberrant when the SNP exhibits an allele frequency that is different from an allele frequency for other SNPs detected on the same segment;
   adjusting, using the one or more processors, the first threshold based on a distribution of aberrant SNP allele frequencies;
   repeating the segmenting, classifying, and adjusting steps when the first threshold is increased; and
   outputting, using the one or more processors, the segmentation data and a final threshold as the estimated degree of contamination for the sample.
38. The method of clause 37, wherein one or more of the plurality of sequence reads overlap one or more gene loci within one or more subgenomic intervals in the sample.
39. The method of clause 37 or clause 38, further comprising setting an initial value for the first threshold as equal to the estimated degree of contamination for the sample.
40. The method of any one of clauses 37 to 39, wherein the plurality of selected single nucleotide polymorphisms (SNPs) comprises a plurality of selected heterozygous single nucleotide polymorphisms (SNPs).
41. The method of any one of clauses 37 to 40, wherein the predetermined distribution of allele frequencies (AFs) for the plurality of selected single nucleotide polymorphisms (SNPs) comprises a predetermined distribution of minor allele frequencies (MAFs) for the plurality of selected single nucleotide polymorphisms (SNPs).
42. The method of clause 37, further comprising using the segmentation data and estimated degree of contamination output by the one or more processors to build a copy number model that predicts a copy number for the one or more gene loci.
43. The method of any one of clauses 37 to 42, further comprising excluding all sequence read data for SNPs that exhibit an allele frequency below the final threshold from a copy number analysis for the one or more gene loci.
44. The method of any one of clauses 37 to 43, further comprising excluding all sequence read data for gene loci on a same segment as SNPs that exhibit an allele frequency below the final threshold from a copy number analysis for the one or more gene loci.
45. The method of any one of clauses 37 to 44, wherein the plurality of selected SNPs identified within the plurality of gene loci comprises at least 100 SNP loci.
46. The method of any one of clauses 37 to 45, wherein the plurality of selected SNPs identified within the plurality of gene loci comprises at least 1,000 SNPs.
47. The method of any one of clauses 37 to 46, wherein the plurality of selected SNPs identified within the plurality of gene loci comprises at most 10,000 SNP loci.
48. The method of any one of clauses 37 to 47, wherein the plurality of selected SNPs identified within the plurality of gene loci comprises at most 100,000 SNP loci.
49. The method of any one of clauses 37 to 48, wherein the plurality of selected SNPs identified within the plurality of gene loci comprise at most 1,000,000 SNP loci.
50. The method of any one of clauses 37 to 49, wherein the plurality of selected single nucleotide polymorphisms (SNPs) identified within a plurality of gene loci comprises biallelic heterozygous SNPs having unbiased heterozygous allele frequencies of about 50%.
51. The method of any one of clauses 37 to 50, wherein the plurality of selected single nucleotide polymorphisms (SNPs) identified within a plurality of gene loci comprises biallelic heterozygous SNPs having reference and alternate alleles that are observed at greater than 20% global allele frequency.
52. The method of clause 51, wherein the plurality of selected single nucleotide polymorphisms (SNPs) identified within a plurality of gene loci comprises biallelic heterozygous SNPs having reference and alternate alleles that are observed at greater than 20% global MAF.
53. The method of any one of clauses 37 to 52, wherein estimating the degree of contamination for the sample based on a distribution of allele frequencies for the plurality of selected SNPs comprises determining a percentage of heterozygous SNPs identified in the sample that have allele frequencies that differ from an expected allele frequency distribution for a plurality of selected heterozygous SNPs identified within the plurality of gene loci by at least a second threshold.
54. The method of any one of clauses 37 to 53, wherein the sequence read data is converted to log 2 coverage ratio data prior to performing the segmenting step.
55. The method of any one of clauses 37 to 54, wherein a SNP is classified as aberrant when the SNP exhibits an allele frequency that is different from the allele frequency for other SNPs detected on the same segment based on an absolute value of the difference in allele frequency.
56. The method of any one of clauses 37 to 55, wherein a SNP is classified as aberrant when the SNP exhibits an allele frequency that is different from the allele frequency for other SNPS detected on the same segment based on a statistical analysis.
57. The method of clause 56, wherein the statistical analysis comprises a t-test.
58. The method of any one of clauses 37 to 57, wherein the segmenting is performed using a circular binary segmentation (CBS) method, a maximum likelihood method, a hidden Markov chain method, a walking Markov method, a Bayesian method, a long-range correlation method, or a change point method.
59. The method of clause 58, wherein the segmenting is performed using a change point method, and the change point method is a pruned exact linear time (PELT) method.
60. The method of any one of clauses 37 to 59, wherein the segmenting, classifying, and adjusting steps are repeated for up to 1 to 10 iterations.
61. The method of any one of clauses 37 to 60, wherein the first threshold is incrementally adjusted to reduce a number of SNPs classified as aberrant, and wherein the first threshold is set based on a percentage of SNPs identified in the sample that have allele frequencies that differ from an expected allele frequency distribution for a plurality of selected heterozygous SNPs identified within the plurality of gene loci by at least a third threshold.
62. The method of any one of clauses 37 to 61, wherein a limit of detection for detecting contamination in the sample is less than about 10%.
63. The method of any one of clauses 37 to 62, wherein a limit of detection for detecting contamination in the sample is less than about 5%.
64. The method of any one of clauses 37 to 63, wherein a limit of detection for detecting contamination in the sample is less than about 1%.
65. The method of any one of clauses 37 to 64, wherein a limit of detection for detecting contamination in the sample is less than about 0.5%.
66. The method of any one of clauses 1 to 65, wherein the first threshold has a value of 0.2, 0.3, 0.4, or 0.5.
67. The method of clause 7 or clause 53, wherein the second threshold is at least 1, at least 2, at least 3, or at least 4 standard deviations from the mean of the expected allele frequency distribution for the plurality of selected heterozygous SNPs.
68. The method of clause 12 or clause 61, wherein the third threshold is at least 1, at least 2, at least 3, or at least 4 standard deviations from the mean of the expected allele frequency distribution for the plurality of selected heterozygous SNPs.
69. A method for calling copy number alterations (CNAs) in a sample from a subject, comprising:
   receiving, at one or more processors, sequence read data for a plurality of sequence reads;
   estimating, using the one or more processors, a degree of contamination for the sample based on a predetermined distribution of allele frequencies (AFs) for a plurality of selected single nucleotide polymorphisms (SNPs) identified within a plurality of gene loci in the sequence read data;
   segmenting, using the one or more processors, the sequence read data into two or more segments, wherein each segment has a same copy number, and wherein sequence read data comprising SNPs that exhibit an allele frequency below a first threshold are excluded from the segmenting process;
   classifying, using the one or more processors, a SNP detected on a segment of the two or more segments as aberrant when the SNP exhibits an allele frequency that is different from an allele frequency for other SNPs detected on the same segment;
   adjusting, using the one or more processors, the first threshold based on a distribution of aberrant SNP allele frequencies;
   repeating the segmenting, classifying, and adjusting steps when the first threshold is increased;
   outputting, using the one or more processors, the segmentation data and a final threshold as the estimated degree of contamination for the sample;
   using the segmentation data and the estimated degree of contamination output by the one or more processors to build a copy number model that predicts a copy number for the one or more gene loci; and
   calling a copy number alterations for the one or more gene loci.
70. The method of clause 69, wherein one or more of the plurality of sequence reads overlap one or more gene loci within one or more subgenomic intervals in the sample.
71. The method of clause 69 or clause 70, further comprising setting an initial value for the first threshold as equal to the estimated degree of contamination for the sample.
72. The method of any one of clauses 69 to 71, wherein the plurality of selected single nucleotide polymorphisms (SNPs) comprises a plurality of selected heterozygous single nucleotide polymorphisms (SNPs).
73. The method of any one of clauses 69 to 72, wherein the predetermined distribution of allele frequencies (AFs) for the plurality of selected single nucleotide polymorphisms (SNPs) comprises a predetermined distribution of minor allele frequencies (MAFs) for the plurality of selected single nucleotide polymorphisms (SNPs).
74. The method of any one of clauses 69 to 73, wherein the called CNAs for the one or more gene loci are used to diagnose or confirm a diagnosis of disease in the subject.
75. The method of clause 74, wherein the disease is cancer.
76. The method of clause 75, further comprising selecting an anti-cancer therapy to administer to the subject based on the called CNAs for the one or more gene loci.
77. The method of clause 76, further comprising determining an effective amount of the anti-cancer therapy to administer to the subject based on the called CNAs for the one or more gene loci.
78. The method of clause 77, further comprising administering the anti-cancer therapy to the subject based on the called CNAs for the one or more gene loci.
79. The method of any one of clauses 75 to 78, wherein the anti-cancer therapy comprises chemotherapy, radiation therapy, immunotherapy, a targeted therapy, or surgery.
80. The method of any one of clauses 75 to 79, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
81. The method of any one of clauses 69 to 80, wherein the one or more gene loci comprise between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
82. A method for diagnosing a disease, the method comprising:
   diagnosing that a subject has the disease based on called CNAs for a sample from the subject, wherein the called CNAs are determined according to the method of any one of clauses 69 to 81.
83. A method of selecting an anti-cancer therapy, the method comprising:
   responsive to calling CNAs for one or more gene loci for a sample from a subject, selecting an anti-cancer therapy for the subject, wherein the called CNAs are is determined according to the method of any one of clauses 69 to 81.
84. A method of treating a cancer in a subject, comprising:
   responsive to calling CNAs for one or more gene loci for a sample from the subject, administering an effective amount of an anti-cancer therapy to the subject, wherein the called CNAs are determined according to the method of any one of clauses 69 to 81.
85. A method for monitoring tumor progression or recurrence in a subject, the method comprising:
   calling CNAs for one or more gene loci in a first sample obtained from the subject at a first time point according to the method of any one of clauses 69 to 81;
   calling CNAs for one or more gene loci in a second sample obtained from the subject at a second time point; and comparing the first called CNAs to the second called CNAs for the one or more gene loci, thereby monitoring the tumor progression or recurrence.
86. The method of clause 85, wherein the called CNAs for the one or more gene loci in the second sample is determined according to the method of any one of clauses 69 to 81.
87. The method of clause 85 or clause 86, further comprising adjusting an anti-cancer therapy in response to the tumor progression.
88. The method of any one of clauses 85 to 87, further comprising adjusting a dosage of the anti-cancer therapy or selecting a different anti-cancer therapy in response to the tumor progression.
89. The method of clause 88, further comprising administering the adjusted anti-cancer therapy to the subject.
90. The method of any one of clauses 85 to 89, wherein the first time point is before the subject has been administered an anti-cancer therapy, and wherein the second time point is after the subject has been administered the anti-cancer therapy.
91. The method of any one of clauses 85 to 90, wherein the subject has a cancer, is at risk of having a cancer, is being routine tested for cancer, or is suspected of having a cancer.
92. The method of any one of clauses 85 to 91, wherein the cancer is a solid tumor.
93. The method of any one of clauses 85 to 91, wherein the cancer is a hematological cancer.
94. The method of any one of clauses 87 to 93, wherein the anti-cancer therapy comprises chemotherapy, radiation therapy, immunotherapy, a targeted therapy, or surgery.
95. The method of any one of clauses 69 to 94, further comprising determining, identifying, or applying the called CNAs for the one or more gene loci in the sample as a diagnostic value associated with the sample.
96. The method of any one of clauses 69 to 95, further comprising generating a genomic profile for the subject based on the called CNAs for the one or more gene loci.
97. The method of clause 96, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
98. The method of clause 96 or clause 97, wherein the genomic profile for the subject further comprises results from a nucleic acid sequencing-based test.
99. The method of any one of clauses 96 to 98, further comprising selecting an anti-cancer agent, administering an anti-cancer agent, or applying an anti-cancer treatment to the subject based on the generated genomic profile.
100. The method of any one of clauses 69 to 99, wherein the called CNAs for the one or more gene loci are used in making suggested treatment decisions for the subject.
101. The method of any one of clauses 69 to 100, wherein the called CNAs for the one or more gene loci are used in applying or administering a treatment to the subject.
102. A system comprising:
   one or more processors; and
   a memory communicatively coupled to the one or more processors and configured to store instructions that, when executed by the one or more processors, cause the system to:
      receive sequence read data for a plurality of sequence reads;
      estimate a degree of contamination for the sample based on a predetermined distribution of allele frequencies (AFs) for a plurality of selected single nucleotide polymorphisms (SNPs) identified within a plurality of gene loci in the sequence read data;
      segment the sequence read data into two or more segments, wherein each segment has a same copy number, and wherein sequence read data comprising SNPs that exhibit an allele frequency below a first threshold are excluded from the segmenting process;
      classify a SNP detected on a segment of the two or more segments as aberrant when the SNP exhibits an allele frequency that is different from an allele frequency for other SNPs detected on the same segment;
      adjust the first threshold based on a distribution of aberrant SNP allele frequencies;
      repeat the segmenting, classifying, and adjusting steps when the first threshold is increased; and
      output the segmentation data and a final threshold as the estimated degree of contamination for the sample.
103. The system of clause 102, wherein the instructions further comprise causing the system to use the segmentation data and estimated degree of contamination to build a copy number model that predicts a copy number for the one or more gene loci.
104. A non-transitory computer-readable storage medium storing one or more programs, the one or more programs comprising instructions, which when executed by one or more processors of a system, cause the system to:
   receive sequence read data for a plurality of sequence reads;
   estimate a degree of contamination for the sample based on a distribution of allele frequencies (AFs) for a plurality of selected single nucleotide polymorphisms (SNPs) identified within a plurality of gene loci in the sequence read data;
   segment the sequence read data into two or more segments, wherein each segment has a same copy number, and wherein sequence read data comprising SNPs that exhibit an allele frequency below a first threshold are excluded from the segmenting process;
   classify a SNP detected on a segment of the two or more segments as aberrant when the SNP exhibits an allele frequence that is different from an allele frequency for other SNPs detected on the same segment;
   adjust the first threshold based on a distribution of aberrant SNP allele frequencies;
   repeat the segmenting, classifying, and adjusting steps when the first threshold is increased; and
   output the segmentation data and a final threshold as the estimated degree of contamination for the sample.
105. The non-transitory computer-readable storage medium of clause 104, wherein the instructions further comprise causing the system to use the segmentation data and estimated degree of contamination to build a copy number model that predicts a copy number for the one or more gene loci.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for detecting contamination in sequence read data for a sample from a subject, the method comprising:
providing a plurality of nucleic acid molecules obtained from the sample from the subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules, wherein one or more of the plurality of sequencing reads overlap one or more gene loci within one or more subgenomic intervals in the sample;
receiving, at one or more processors, sequence read data for the plurality of sequence reads;
estimating, using the one or more processors, a degree of contamination for the sample based on a predetermined distribution of allele frequencies (AFs) for a plurality of selected single nucleotide polymorphisms (SNPs) identified within a plurality of gene loci in the sequence read data;
segmenting, using the one or more processors, the sequence read data into two or more segments, wherein each segment has a same copy number, and wherein sequence read data comprising SNPs that exhibit an allele frequency below a first threshold are excluded from the segmenting process;
classifying, using the one or more processors, a SNP detected on a segment of the two or more segments as aberrant when the SNP exhibits an allele frequency that is different from an allele frequency for other SNPs detected on the same segment;
adjusting, using the one or more processors, the first threshold based on a distribution of aberrant SNP allele frequencies;
repeating the segmenting, classifying, and adjusting steps when the first threshold is increased; and
outputting, using the one or more processors, the segmentation data and a final threshold as the estimated degree of contamination for the sample.

2. (canceled)

3. The method of claim 1, further comprising setting an initial value for the first threshold as equal to the estimated degree of contamination for the sample.

4. The method of claim 1, wherein the plurality of selected single nucleotide polymorphisms (SNPs) comprises a plurality of selected heterozygous single nucleotide polymorphisms (SNPs).

5. The method of claim 1, wherein the predetermined distribution of allele frequencies (AFs) for the plurality of selected single nucleotide polymorphisms (SNPs) comprises a predetermined distribution of minor allele frequencies (MAFs) for the plurality of selected single nucleotide polymorphisms (SNPs).

6. The method of claim 1, further comprising using the segmentation data and estimated degree of contamination output by the one or more processors to build a copy number model that predicts a copy number for the one or more gene loci.

7. The method of claim 1, further comprising excluding all sequence read data for SNPs that exhibit an allele frequency below the final threshold from a copy number analysis for the one or more gene loci.

8. The method of claim 1, further comprising excluding all sequence read data for gene loci on a same segment as SNPs that exhibit an allele frequency below the final threshold from a copy number analysis for the one or more gene loci.

9. The method of claim 1, wherein the plurality of selected single nucleotide polymorphisms (SNPs) identified within the plurality of gene loci comprises at least 1,000 SNPs.

10. The method of claim 1, wherein the plurality of selected single nucleotide polymorphisms (SNPs) identified within a plurality of gene loci comprises biallelic heterozygous SNPs having unbiased heterozygous allele frequencies of about 50%.

11. The method of claim 1, wherein the plurality of selected single nucleotide polymorphisms (SNPs) identified within a plurality of gene loci comprises biallelic heterozygous SNPs having reference and alternate alleles that are observed at greater than 20% global allele frequency.

12. The method of claim 11, wherein the plurality of selected single nucleotide polymorphisms (SNPs) identified within a plurality of gene loci comprises biallelic heterozygous SNPs having reference and alternate alleles that are observed at greater than 20% global MAF.

13. The method of claim 1, wherein estimating the degree of contamination for the sample based on a distribution of allele frequencies for the plurality of selected SNPs comprises determining a percentage of heterozygous SNPs identified in the sample that have allele frequencies that differ from an expected allele frequency distribution for a plurality of selected heterozygous SNPs identified within the plurality of gene loci by at least a second threshold.

14. The method of claim 1, wherein the sequence read data is converted to log 2 coverage ratio data prior to performing the segmenting step.

15. The method of claim 1, wherein a SNP is classified as aberrant when the SNP exhibits an allele frequency that is different from the allele frequency for other SNPs detected on the same segment based on an absolute value of the difference in allele frequency.

16. The method of claim 1, wherein a SNP is classified as aberrant when the SNP exhibits an allele frequency that is different from the allele frequency for other SNPS detected on the same segment based on a statistical analysis.

17. (canceled)

18. The method of claim 1, wherein the segmenting is performed using a circular binary segmentation (CBS) method, a maximum likelihood method, a hidden Markov chain method, a walking Markov method, a Bayesian method, a long-range correlation method, or a change point method.

19. The method of claim 18, wherein the segmenting is performed using a change point method, and the change point method is a pruned exact linear time (PELT) method.

20. The method of claim 1, wherein the segmenting, classifying, and adjusting steps are repeated for up to 1 to 10 iterations.

21. The method of claim 1, wherein the first threshold is incrementally adjusted to reduce a number of SNPs classified as aberrant, and wherein the first threshold is set based on a percentage of SNPs identified in the sample that have allele frequencies that differ from an expected allele frequency distribution for a plurality of selected heterozygous SNPs identified within the plurality of gene loci by at least a third threshold.

22. The method of claim 1, wherein a limit of detection for detecting contamination in the sample is less than about 5%.

23. The method of claim 1, wherein the first threshold has a value of 0.2, 0.3, 0.4, or 0.5.

24. The method of claim 13, wherein the second threshold is at least 1, at least 2, at least 3, or at least 4 standard deviations from the mean of the expected allele frequency distribution for the plurality of selected heterozygous SNPs.

25. The method of claim 21, wherein the third threshold is at least 1, at least 2, at least 3, or at least 4 standard deviations from the mean of the expected allele frequency distribution for the plurality of selected heterozygous SNPs.

26. A method for calling copy number alterations (CNAs) in a sample from a subject, comprising:
receiving, at one or more processors, sequence read data for a plurality of sequence reads, wherein one or more of the plurality of sequence reads overlap one or more gene loci within one or more subgenomic intervals in the sample;
estimating, using the one or more processors, a degree of contamination for the sample based on a predetermined distribution of allele frequencies (AFs) for a plurality of selected single nucleotide polymorphisms (SNPs) identified within a plurality of gene loci in the sequence read data;
segmenting, using the one or more processors, the sequence read data into two or more segments, wherein each segment has a same copy number, and wherein sequence read data comprising SNPs that exhibit an allele frequency below a first threshold are excluded from the segmenting process;
classifying, using the one or more processors, a SNP detected on a segment of the two or more segments as aberrant when the SNP exhibits an allele frequency that is different from an allele frequency for other SNPs detected on the same segment;
adjusting, using the one or more processors, the first threshold based on a distribution of aberrant SNP allele frequencies;
repeating the segmenting, classifying, and adjusting steps when the first threshold is increased;
outputting, using the one or more processors, the segmentation data and a final threshold as the estimated degree of contamination for the sample;
using the segmentation data and the estimated degree of contamination output by the one or more processors to build a copy number model that predicts a copy number for the one or more gene loci; and
calling a copy number alterations for the one or more gene loci.

27. (canceled)

28. The method of claim 26, further comprising setting an initial value for the first threshold as equal to the estimated degree of contamination for the sample.

29. The method of claim 26, wherein the plurality of selected single nucleotide polymorphisms (SNPs) comprises a plurality of selected heterozygous single nucleotide polymorphisms (SNPs).

30. The method of claim 26, wherein the predetermined distribution of allele frequencies (AFs) for the plurality of selected single nucleotide polymorphisms (SNPs) comprises a predetermined distribution of minor allele frequencies (MAFs) for the plurality of selected single nucleotide polymorphisms (SNPs).

31. The method of claim 26, wherein the called CNAs for the one or more gene loci are used to diagnose or confirm a diagnosis of disease in the subject.

32. The method of claim 31, wherein the disease is cancer.

33. The method of claim 32, further comprising selecting an anti-cancer therapy to administer to the subject based on the called CNAs for the one or more gene loci.

34. The method of claim 33, further comprising determining an effective amount of the anti-cancer therapy to administer to the subject based on the called CNAs for the one or more gene loci.

35. The method of claim 34, further comprising administering the anti-cancer therapy to the subject based on the called CNAs for the one or more gene loci.

36. The method of claim 32, wherein the anti-cancer therapy comprises chemotherapy, radiation therapy, immunotherapy, a targeted therapy, or surgery.

37.-40. (canceled)
